(19) United States
(12) Patent Application Publication
(10) Pub. No.: US 2004/0063742 A1
(43) Pub. Date: Apr. 1, 2004

(54) Treatment of parkinson's disease by the combined action of a compound with neurotrophic activity and a compound enhancing the dopamine activity

(76) Inventors: Dan Peters, Malmo (DK); Lars Christian Ronn, Vekso (DK); Gunnar M. Olsen, Frederiksberg (DK); Arne Moller, Sjaellands Odde (DK)

Correspondence Address: BIRCH STEWART KOLASCH & BIRCH, PO BOX 747, FALLS CHURCH, VA, 22040-0747, US

(21) Appl. No.: 10/466,435

(22) PCT Filed: Feb. 15, 2002
(86) PCT No.: PCT/DK02/00107

(30) Foreign Application Priority Data
Feb. 15, 2001 (DK) PA 2001 00255

(51) Int. Cl. A61K 31/4745; A61K 31/403

(52) U.S. Cl. 514/291; 514/411; 546/81; 548/427

(57) ABSTRACT

This invention relates to the use of the combined action of a compound with neurotrophic activity and a compound enhancing the dopamine activity for the treatment of Parkinson's disease.

### TECHNICAL FIELD

This invention relates to the use of the combined action of a compound with neurotrophic activity and a compound enhancing the dopamine activity for the treatment of Parkinson's disease.

### BACKGROUND ART

Parkinson's disease is a neurodegenerative disease characterised by the progressive deterioration of motor skills, affecting about 4 million people worldwide. Parkinson's patients suffer from increasing difficulties in initiating movement, rigidity in arms and legs, as well as tremors. Although the specific cause of Parkinson's disease is unknown, it has been shown that the disease is associated with the degeneration of specific dopamine-containing neurons in a region of the brain known as the substanta nigra, which is believed to be involved in the coordination of movement.

One existing treatment is L-DOPA therapy, alone or combined with e.g. dopamine agonists. However, after three to five years of L-DOPA therapy, involuntary motor disturbances (dyskinesia) may appear.

Another treatment is the use of monoamine reuptake inhibitors (such as dopamine reuptake inhibitors) whereby the existing dopamine level in the synaptic cleft is increased.

A further possible therapy is the use of neurotrophic compounds which give a neuroprotective and/or neuroregenerative effect on lesioned and damaged neurons.

There is a continued strong interest in the development of a more selective and effective therapy with fewer side effects for the treatment of patients with Parkinson's disease.

### SUMMARY OF THE INVENTION

According to the invention it has now been found that the action of a compound with neurotrophic activity in combination with a compound enhancing the dopamine activity advantageously can be used for the treatment of Parkinson's dis ase.

Accordingly, in its first aspect, the invention related to a pharmaceutical composition comprising a th rapeutically effective amount of at least one compound with neurotrophic activity and at least one compound enhancing the dopamine activity, together with at least one pharmaceutically acceptable carrier or diluent.

In another aspect, the invention relates to the use of at least one compound with neurotrophic activity and at least one compound enhancing the dopamine activity for the manufacture of a medicament for the treatment, prevention or alleviation of Parkinson's disease in a subject.

In a further aspect, the invention relates to novel compounds having neurotrophic activity.

In a still further aspect, the invention relates to methods for screening and for identification of a compound for activity in the treatment, prevention or alleviation of Parkinson's disease in a subject.

The principle combines a fast onset action (the enhanced dopamine activity) with a long-term effective principle (the neurotrophic activity). Thus, the enhanced dopamine activity relieves the symptoms of the disease (low dopamine release), while the neurotrophic activity treats the cause of the disease (degenerating neurons).

Other objects of the invention will be apparent to the person skilled in the art from the following detailed description and examples.

### DETAILED DISCLOSURE OF THE INVENTION

In its first aspect, the invention provides a pharmaceutical composition comprising a therapeutically effective amount of at least one compound with neurotrophic activity and at least one compound enhancing the dopamine activity, together with at least one pharmaceutically acceptable carrier or diluent.

In a second aspect, the invention provides a combination of at least one compound with neurotrophic activity and at least one compound enhancing the dopamine activity for use as a therapeutic agent.

In a third aspect, the invention provides the use of at least one compound with neurotrophic activity and at least one compound enhancing the dopamine activity for the manufacture of a medicament for the treatment, prevention or alleviation of Parkinson's disease in a subject.

In a further aspect, the invention provides a method of treatment, prevention or alleviation of Parkinson's disease in a subject, which method comprises administering to said subject a therapeutically effective combination of at least one compound with neurotrophic activity and at least one compound enhancing the dopamine activity.

In a still further aspect, the invention provides a kit of parts comprising at least one compound with neurotrophic activity and at least one compound enhancing the dopamine activity.

In a further aspect, the invention relates to a novel compound being

[N-(5-Chloro-2-hydroxyphenyl)-N′-(3-nitrophenyl)]urea;

[N-(2-Hydroxy-4-methoxyphenyl)-N′-phenyl]urea;

[N-(3-Aminophenyl)-N′-(2-hydroxy-4-methoxyphenyl)]urea;

[N-(3-Chlorophenyl)-N′-(2-hydroxy-4-methoxyphenyl)]urea;

[N-(2-Hydroxy-4-methoxyphenyl)-N′-(4-trifluoromethylphenyl)]urea;

[N-(2-Hydroxy-4-methoxyphenyl)-N′-(3-nitrophenyl)]urea;

[N-(4-Chlorophenyl)-N′-(2-hydroxy-4-methoxyphenyl)]urea;

[N-(2-Hydroxy-4-methoxyphenyl)-N′-(2-trifluoromethylphenyl)]urea;

[N-(4-Chloro-2-hydroxy-5-methylphenyl)-N′-(phenyl)]urea;

[N-(3-Bromophenyl)-N′-(2-hydroxy-4-methoxyphenyl)]urea;

[N-(3-Fluorophenyl)-N′-(2-hydroxy-4-methoxyphenyl)]urea;

[N-(2-Chloro-5-trifluoromethylphenyl)-N′-(2-hydroxy-4-methoxyphenyl)]urea;

[N-(3,5-Di-(trifluoromethyl)-phenyl)-N′-(2-hydroxy-4-methoxyphenyl)]urea;

[N-(5-Bromo-2-hydroxyphenyl)-N′-(3-trifluoromethylphenyl)]urea;

[N-(2-Hydroxy-4-methoxyphenyl)-N′-(3-methoxycarbonylphenyl)]urea;

[N-(2-Hydroxy-4-methoxyphenyl)-N′-(3-methylcarbonylphenyl)]urea;

[N-(3-Cyanophenyl)-N′-(2-hydroxy-4-methoxyphenyl)]urea;

[N-(5-Ethoxy-2-hydroxy-4-(N-morpholinyl)phenyl)-N′-(3-trifluoromethylphenyl)]urea;

or pharmaceutically acceptable salts thereof.

In a still further aspect, the invention relates to a method for screening a compound for activity in the treatment, prevention or alleviation of Parkinson's disease in a subject, which method comprises the following steps:

measuring the ability of the compound to enhance the dopamine activity;

measuring the neurotrophic activity of the compound.

In a further aspect, the invention relates to a method for identification of a compound active in the treatment, prevention or alleviation of Parkinson's disease in a subject, which method comprises the following steps:

screening compounds for the ability to enhance the dopamine activity and for the neurotrophic activity:

selecting a compound with ability to enhance the dopamine activity and with neurotrophic activity.

In one embodiment, the compound enhancing the dopamine activity is a monoamine reuptake Inhibitor. In a second embodiment, the compound enhancing the dopamine activity Is a dopamine agonist.

In a second embodiment, the compound with neurotrophic activity is a compound selected from the novel compounds listed above.

In a third embodiment, the compound with neurotrophic activity is a compound selected from

[N-(2-Hydroxy-4-methoxyphenyl)-N′-(3-trifluoromethylphenyl)]urea;

5-(4-Chlorophenyl)-8-methyl-6,7,8,9-tetrahydro-1-H-pyrrolo[3.2-h]isoquinoline-2,3-dione-3-oxime;

5-(4-Chlorophenyl)-6,7,8,9-tetrahydro-1-H-pyrrolo[3.2-h]naphthalene-2,3-dione-3-oxime;

5-(4-Chlorophenyl)-6,7,8,9-tetrahydro-1-H-pyrrolo[3.2-h]naphthalene-2,3-dione-3-O-(4-hydroxybutyric acid-2-yl)oxime;

or pharmaceutically acceptable salts thereof.

In a further embodiment, the compound with neurotrophic activity and the compound enhancing the dopamine activity are not the same compound. In a special embodiment, the compound with neurotrophic activity is GDNF and the compound enhancing the dopamine activity is Bupropion. In a further special embodiment, the compound with neurotrophic activity is GDNF and the compound enhancing the dopamine activity is Nomifensine.

In a still further embodiment, the compound with neurotrophic activity and the compound enhancing the dopamine activity are the same compound. In a special embodiment, the compound with neurotrophic activity and the compound enhancing the dopamine activity are selected from

5-(4-Chlorophenyl)-8-methyl-6,7,8,9-tetrahydro-1-H-pyrrolo[3.2-h]isoquinoline-2,3-dione-3-oxime;

5-(4-Chlorophenyl)-6,7,8,9-tetrahydro-1-H-pyrrolo[3.2-h]naphthalene-2,3-dione-3-oxime; 5-(4-Chlorophenyl)-6,7,8,9-tetrahydro-1-H-pyrrolo[3.2-h]naphthalene-2,3-dione-3-O-(4-hydroxybutyric acid-2-yl)oxime;

and pharmaceutically acceptable salts thereof.

In a further embodiment, the pharmaceutical composition as described above is for use in the treatment, prevention or alleviation of a neurodegenerative condition. In a still further embodiment, the pharmaceutical composition as described above is for use in the treatment, prevention or alleviation of Parkinson's disease in a subject.

The subject to be treated according to this invention is a living body, preferably a mammal, most preferably a human, in need for such treatment.

Compounds with Neurotr phic Activity

Endogenous neurotrophic factors, such as nerve growth factor (NGF), brain-derived neurotrophic factor (BDNF), epidermal growth factor (EGF), basic fibroblast growth factor (bFGF, or FGF2), NT3/4, neurturin (NTN), neublastin/artemin, persephin, and glial cell-line derived neurotrophic factor (GDNF), promote the differentiation, growth and survival of numerous peripheral and central nervous system neurons during development and adulthood.

In the context of this invention, compounds with neurotrophic activity are compounds that mimic or enhance the function of one or more endogenous neurotrophic factors. In one embodiment, a compound with neurotrophic activity is a compound that mimics or enhances the function of NGF, BDNF, and/or GDNF. In a further embodiment, a compound with neurotrophic activity is a compound that mimics or enhances the function of bFGF and/or EGF. In a special embodiment, a compound with neurotrophic activity is a compound that mimics or enhances the function of NGF. The neurotrophic activity has not been ascribed to a specific step in the interaction between NGF and its receptor or in the NGF signal transduction pathway.

The potential of a given substance to act as a compound with neurotrophic activity may be determined using standard in vitro binding assays and/or standard in vivo functional tests, such as those described in “Test methods”.

In one embodiment, the compound with neurotrophic activity at 1 μM shows more than 10% (more preferably more than 20%, and most preferably more than 30%) of the effect of 3 nM NGF when tested in the PC12 cells survival assay (method 2).

Compounds with neurotrophic activities for use according to the invention include those substances described in the patent applications WO 98/07705 (Takeda Chem Ind Ltd), WO 00/34262 (Takeda Chem Ind Ltd), WO00/32197 (Alcon Lab Inc), WO 97/40035 (NeuroSearch), WO 00/43397 (NeuroSearch), WO 01/55110 (NeuroSearch), JP2000226388-A (Takeda Chem Ind Ltd), WO00/32197 (Alcon Lab), and WO 00/46222 (Schering AG).

Further examples of compounds with neurotrophic activity according to the invention include 1-(1,3-benzodioxol-5-yl)-7,8,9,10-tetrahydro-1,3-benzodioxol[4,5-g]isoquinolin-7-one (Takeda), 2-(2,2,4,6,7-Pentamethyl-3-phenyl-2,3-dihydro-1-benzofuran-5-yl)-isoindoline (Takeda), 4-Aryl-1-phenylalkyl-1,2,3,6-tetrahydropyridine (Sanofi-Synthelabo), SR 57746A or 1-(2-napht-2-yl)ethyl-4-(3-trifluoromethylphenyl)-1,2,5,6-tetrahydropyridine (Sanofi-Synthelabo), AIT-082 (NeoTherapeutics), NIL-A (Amgen Inc), K-252a (Cephalon), CEP 1347, GPI-1046 (Guilford), CTQ3, CTQ5 and CTQ8 (Centre de Neurochimie du CNRS), V-10,367 and V-13,661 (Vertex Pharmaceuticals Inc), ABS 205 (American Biogenic Sciences), Dexanabinol or HU-211 (Pharmos), or salts, free bases, racemates or enantiomers thereof.

The above examples of compounds with neurotrophic activity are not intended to be in any way limiting to the scope of the invention as claimed.

Substances Enhancing the Dopamine Activity

In the context of the present invention, substances enhancing the dopamine activity include L-DOPA, monoamine oxidase inhibitors, dopamine agonists, and monoamine reuptake inhibitors.

The potential of a given substance to enhance the dopamine activity may be determined using standard in vitro binding assays and/or standard in vivo functionality tests, such as those described in “Test methods”.

L-DODA may be used in combination with decarboxylase inhibitors (such as carbidopa) or COMT inhibitors (such as entacapone).

Examples of monoamine oxidase inhibitors are monoamine oxidase B inhibitors, such as selegiline.

Examples of dopamine agonists are bromocriptine, pergolide, cabergoline, ropinirole, pramipexole, or apomorphine in combination with domperidone.

The monoamine reuptake inhibitor for use according to the invention may in particular be a mixed monoamine reuptake inhibitor, a noradrenaline/dopamine uptake inhibitor, a classical tricyclic antidepressive agent, a selective dopamine reuptake inhibitor, or a relatively selective dopamine reuptake inhibitor.

In one embodiment, the monoamine reuptake inhibitor show an IC₅₀ value of less than 10 μM, preferably less than 1 μM, and most preferably less than 0.1 μM, when tested for in vitro inhibition of ³H-DA uptake (test method 8).

Examples of mixed monoamine reuptake inhibiting drugs include those described in WO 97/16451 (NeuroSearch) and WO 97/13770 (NeuroSearch). The most preferred mixed monoamine reuptake inhibiting drugs include (1S,3S,4S,5S,8R)-3-(3,4-dichlorophenyl)-7-azatricyclo[5.3.0.0]decan-5-ol.

Examples of NA/DA-uptake inhibitors include drugs like Venlafaxin, Minacipram, Reboxetin.

Examples of classic tricyclic antidepressiva include drugs like Imipramin, Amitriptyline, Clomipramine, Doxepin, Amoxapine, Desipramine, Maprotiline, Nortriptyline and Protriptyline.

Examples of selective dopamine reuptake inhibitors include GRB-12909, GRB-12935, Indatraline (Lu-19-005), Bupropion, Amfonelic acid, BTCP, Mazindol, Nomifensine, Beta-CFT (WIN 35,428), Beta-CTP (WIN 35,065-2), Beta-CIT (RTI-55), GYKI 52895, 4′,4″-Diflouro-3-alpha-diphenyl-methoxytropane, 4′-Chloro-3-alpha-diphenylmethoxytropane, 5-(4-Chlorophenyl)-8-methyl-6,7,8,9-tetrahydro-1-H-pyrrolo[3.2-h]isoquinoline-2,3-dione-3-oxime; and 5-(4-Chlorophenyl)-6,7,8,9-tetrahydro-1-H-pyrrolo[3.2-h]naphthalene-2,3-dione-3-oxime;.

Examples of relatively selective dopamine reuptake inhibitors include amineptine, 3,4-dichlorophenyl 4-(3,4-dichlorophenyl)-4-hydroxy-1-methyl-3-piperidyl ketone (Wang, S et al, 1999), 1-[2-(diphenylmethoxy)ethyl]-4-(3-phenylpropyl)homopiperazine (LR-1111), 1-[2-(diphenylmethoxy)-ethyl]-4-(3-phenyl-2-propenyl-)homopiperazine, (S)-(−)-1-[2-(diphenylmethoxy)ethyl]-2-[[N-(3-phenylpropyl)amino]methyl]pyrrolidine, and (S)-(−)-1-[2-[bis(4-fluorophenyl)-methoxy]ethyl]-2-[[N-(3-phenylpropyl)amino]methyl]pyrrolidine.

Monoamine reuptake inhibitors for use according to the invention also include ALE-26018 and the dopamine reuptake inhibitors as described in the patents US6011070, US5821386, US6001330, US5795915, and US5574060.

In a special embodiment, the substance enhancing the dopamine activity is selected from GRB-12909, GRB-12935, and 5-(4-Chlorophenyl)-6,7,8,9-tetrahydro-1-H-pyrrolo[3.2-h] naphthalene-2,3-dione-3-oxime;

The above examples of compounds enhancing the dopamine activity are not intended to be in any way limiting to the scope of the invention as claimed.

Novel Compounds

The novel compounds of the invention may be prepared by conventional methods for chemical synthesis. All N,N′-diarylureas were prepared by mixing the corresponding arylurea and arylisocyanate in toluene.

The end products of the reactions described herein may be isolated by conventional techniques, e.g. by extraction, crystallisation, distillation, chromatography, etc.

The following novel compounds were prepared:

[N-(2-Hydroxy-4-methoxyphenyl)-N′-(3-trifluoromethylphenyl)]urea, mp 170-171° C.

[N-(5-Chloro-2-hydroxyphenyl)-N′-(3-nitrophenyl)]urea, mp 171-174° C.

[N-(2-Hydroxy-4-methoxyphenyl)-N′-phenyl]urea, mp 157-159° C.

[N-(3-Aminophenyl)-N′-(2-hydroxy-4-methoxyphenyl)]urea, mp 164-165° C.

[N-(3-Chlorophenyl)-N′-(2-hydroxy-4-methoxyphenyl)]urea, mp 162-165° C.

[N-(2-Hydroxy-4-methoxyphenyl)-N′-(4-trifluoromethylphenyl)]urea, mp 192-193° C.

[N-(2-Hydroxy-4-methoxyphenyl)-N′-(3-nitrophenyl)]urea, mp 177-179° C.

[N-(4-Chlorophenyl)-N′-(2-hydroxy-4-methoxyphenyl)]urea, mp 187-188° C.

[N-(2-Hydroxy-4-methoxyphenyl)-N′-(2-trifluoromethylphenyl)]urea, mp 165-167° C.

[N-(4-Chloro-2-hydroxy-5-methylphenyl)-N′-(phenyl)]urea, mp 180-181° C.

[N-(3-Bromophenyl)-N′-(2-hydroxy4-methoxyphenyl)]urea, mp 186-187° C.

[N-(3-Fluorophenyl)-N′-(2-hydroxy-4-methoxyphenyl)]urea, mp 167-169° C.

[N-(2-Chloro-5-trifluoromethylphenyl)-N′-(2-hydroxy-4-methoxyphenyl)]urea, mp 156-158° C.

[N-(3,5-Di-(trifluoromethyl)-phenyl)-N′-(2-hydroxy-4-methoxyphenyl)]urea, mp 177-179° C.

[N-(5-Bromo-2-hydroxyphenyl)-N′-(3-trifluoromethylphenyl)]urea, 164-166° C.

[N-(2-Hydroxy-4-methoxyphenyl)-N′-(3-methoxycarbonylphenyl)]urea, 165-167° C.

[N-(2-Hydroxy-4-methoxyphenyl)-N′-(3-methylcarbonylphenyl)]urea.

[N-(3-Cyanophenyl)-N′-(2-hydroxy-4-methoxyphenyl)]urea, mp 188-190° C.

[N-(5-Ethoxy-2-hydroxy-4-(N-morphollnyl)phenyl)-N′-(3-trifluoromethylphenyl)]urea, mp 234-236° C.

Pharmaceutically Acceptable Salts

The chemical compound of the invention may be provided in any form suitable for the intended administration. Suitable forms include pharmaceutically (i.e. physiologically) acceptable salts, and pre- or prodrug forms of the chemical compound of the invention.

Examples of pharmaceutically acceptable addition salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulphonate derived from benzensulphonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Such salts may be formed by procedures well known and described in the art.

Other acids such as oxalic acid, which may not be considered pharmaceutically acceptable, may be useful in the preparation of salts useful as intermediates in obtaining a chemical compound of the invention and its pharmaceutically acceptable acid addition salt.

Metal salts of a chemical compound of the invention includes alkali metal salts, such as the sodium salt of a chemical compound of the invention containing a carboxy group.

In the context of this invention the “onium salts” of N-containing compounds are also contemplated as pharmaceutically acceptabl salts. Preferred “onium salts” include the alkyl-onium salts, the cycloalkyl-onium salts, and the cycloalkylalkyl-onium salts.

The chemical compound of the invention may be provided in dissoluble or indissoluble forms together with a pharmaceutically acceptable solvents such as water, ethanol, and the like. Dissoluble forms may also include hydrated forms such as the monohydrate, the dihydrate, the hemihydrate, the trihydrate, the tetrahydrate, and the like. In general, the dissoluble forms are considered equivalent to indissoluble forms for the purposes of this invention.

Prodrugs

The chemical compound of the invention may be administered as such or in the form of a suitable prodrug.

The term “prodrug” denotes a compound, which is a drug precursor and which, following administration and absorption, release the drug in vivo via some metabolic process.

Particularly favoured prodrugs are those that increase the bioavailability of the compounds of the invention (e.g. by allowing an orally administrered compound to be more readily absorbed into the blood) or which enhance delivery of the parent compound to a specific biological compartment (e.g. the brain or lymphatic system).

Thus examples of suitable prodrugs of the substances according to the invention include compounds modified at one or more reactive or derivatizable groups of the parent compound. Of particular interest are compounds modified at a carboxyl group, a hydroxyl group, or an amino group. Examples of suitable derivatives are esters or amides.

Steric Isomers

The chemical compounds of the present invention may exist in (+) and (−) forms as well as in racemic forms. The racemates of these isomers and the individual isomers themselves are within the scope of the present invention.

Racemic forms can be resolved into the optical antipodes by known methods and techniques. One way of separating the diastereomeric salts is by use of an optically active acid, and liberating the optically active amine compound by treatment with a base. Another method for resolving racemates into the optical antipodes is based upon chromatography on an optical active matrix. Racemic compounds of the present invention can thus be resolved into their optical antipodes, e.g., by fractional crystallisation of d- or I- (tartrates, mandelates, or camphorsulphonate) salts for example.

The chemical compounds of the present invention may also be resolved by the formation of diastereomeric amides by reaction of the chemical compounds of the present invention with an optically active activated carboxylic acid such as that derived from (+) or (−) phenylalanine, (+) or (−) phenylglycine, (+) or (−) camphanic acid or by the formation of diastereomeric carbamates by reaction of the chemical compound of the present invention with an optically active chloroformate or the like.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optical active starting materials.

Pharmaceutical Compositions

While a chemical compound of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the chemical compound of the invention, or a pharmaceutically acceptable salt or derivative thereof, together with one or more pharmaceutically acceptable carriers therefor, and, optionally, other therapeutic and/or prophylactic ingredients. The carrier(s) must be “acceptable” in the sense of being compatible with the other ingredients of the formulation and not harmful to the recipient thereof.

Pharmaceutical compositions of the invention may be those suitable for oral, rectal, bronchial, nasal, topical (including buccal and sub-lingual), transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

The chemical compound of the invention, together with a conventional adjuvant, carrier, or diluent, may thus be placed into the form of pharmaceutical compositions and unit dosages thereof. Such forms include solids, and in particular tablets, filled capsules, powd r and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The chemical compound of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid, which is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as a mixture of fatty acid glyceride or cocoa butter, is first melted and the active component is dispersed homogeneously therein, as by stirring. The molten homogenous mixture is then poured into convenient sized moulds, allowed to cool, and thereby to solidify.

Compositions suitable for vaginal administration may be presented as pessaries, tampons, creams, gels, past s, foams or sprays containing in addition to the active ingredient such carriers as are known in the art to be appropriate.

Liquid preparations include solutions, suspensions, and emulsions, for example, water or water-propylene glycol solutions. For example, parenteral injection liquid preparations can be formulated as solutions in aqueous polyethylene glycol solution.

The chemical compound according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilising and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations, intended for conversion shortly before use to liquid form preparations for oral administration. Such liquid forms include solutions, suspensions, and emulsions. In addition to the active component such preparations may comprise colorants, flavours, stabilisers, buffers, artificial and natural sweeteners, dispersants, thickeners, solubilizing agents, and the like.

For topical administration to the epidermis the chemical compound of the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Compositions suitable for topical administration in the mouth include lozenges comprising the active agent in a flavoured base, usually sucrose and acacia or tragacanth; pastilles comprising the active ingredient in an inert base such as gelatin and glycerine or sucrose and acacia; and mouthwashes comprising the active ingr dient in a suitable liquid carrier.

Solutions or suspensions are applied directly to the nasal cavity by conventional means, for example with a dropper, pipette or spray. The compositions may be provided in single or multi-dose form.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dlchlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

In compositions intended for administration to the respiratory tract, including intranasal compositions, the compound will generally have a small particle size for example of the order of 5 microns or less. Such a particle size may be obtained by means known in the art, for example by micronization.

When desired, compositions adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration and continuous infusion are preferred compositions.

Further details on techniques for formulation and administration may be found in the latest edition of Remington's Pharmaceutical Sciences (Maack Publishing Co., Easton, Pa.).

A therapeutically effective dose refers to that amount of active ingredient, which ameliorates the symptoms or condition. Therapeutic efficacy and toxicity, e.g. ED₅₀ and LD₅₀, may be determined by standard pharmacological procedures in cell cultures or experimental animals. The dose ratio between therapeutic and toxic effects is the therapeutic Index and may be expressed by the ratio LD₅/ED₅₀. Pharmaceutical compositions exhibiting large therapeutic indexes are preferred.

The dose administered must of course be carefully adjusted to the age, weight and condition of the individual being treated, as well as the route of administration, dosage form and regimen, and the result desired, and the exact dosage should of course be determined by the practitioner.

The actual dosage depend on the nature and severity of the disease being treated and the route of administration, and is within the discretion of the physician, and may be varied by titration of the dosage to the particular circumstances of this invention to produce the desired therapeutic effect. However, it is presently contemplated that pharmaceutical compositions containing of from about 0.01 to about 500 mg of active ingredient per individual dose, preferably of from about 0.1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.01 μg/kg i.v. and 0.1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Test Methods

Method 1

Stimulation of Neurite Outgrowth in PC12 Cells

In this test, the ability of a compound with neurotrophic activity (below: the compound) to potentiate NGF-induced neurite outgrowth in PC12 cells is assessed.

Method

PC12 cells are seeded in tissue culture plates coated with collagen at a cell density of 15,000/cm² in DMEM with 7.5% FCS and 7.5% DHS. Next day the medium is changed to medium supplemented with the compound in the absence or presence of NGF.

Two days after the medium change, cells are fixed in 4% paraformaldehyde and stained for neurofilament. Cells are fixed by in tissue culture plates by incubation in 4% paraformaldehyde in PBS, followed by permeabilization in 0.05% Triton-X100 in the presence of 10% DHS to block non-specific binding sites. After washing, the plates are incubated with anti-neurofilament (NF) antibody (clone RT97, Boehringer) diluted 1:200 in 0.05% Triton-X100/10% DHS followed by incubation with biotinylated anti-mouse immunoglobulin RPN1001 (Amersham) diluted 1:200. NF-immunoreactive cells are stained using the ABC-complex/HRP kit K0355 (DAKO) and 3,3-diaminobenzidine tetrahydrochloride (DAB) as substrate.

Estimation of total cell number per well, as well as the total neurite length are done using unbiased 2D stereology (CAST-grid system connected to a Olympus BH-2 microscope).

Method 2

PC12 Cells Survival Assay

In this test, the effect of a compound with neurotrophic activity (below: the compound) on the survival of PC12 cells is assessed.

Method

PC12 cells are seeded in collagen-coated 96 well plates in growth medium supplemented with 2 nM mouse 7S NGF (Alomone Labs Ltd., Jerusalem, Israel) and cultured for 6 days. The medium is then changed to serum-free DMEM supplemented with the compound. NGF (3 nM) is included as a positive control. After 4 days of incubation, cell viability is evaluated by using the CyQUANT Cell Proliferation assay according to the manufacturer's instructions (Molecular Probes, C-7026). Briefly, medium is aspirated, and cells are incubated at −80° C. for at least 1 hour. Cells are then thawed and incubated in a buffer containing the fluorescent CyQUANT dye, which exhibits strong fluorescence enhancement when bound to nucleic acids. Fluorescence measured with excitation at 480 nm and emission detection af 520 nm can be correlated to the number of living cells in the wells.

Method 3

Survival of Embryonic Rat Dopaminergic Neurons

In this test, the effect of a compound with neurotrophic activity (below: the compound) on the survival of dopaminergic neurons in dissociated cultures established from rat E14 ventral mesencephali (VM) is assessed.

Method

Embryonic rat brains (Wistar; E14) are isolated under sterile conditions placed in chilled Gey's balanced salt solution (GIBCO) with glucose (6.5 mg/ml).

The ventral mesencephali are dissected out, cut into small tissue pieces, placed in Neurobasal medium with B27 supplement and gently pressed through a 80 μm Nitex filter. The cells are counted using a hemocytometer and plated in a 6 well multi-dish at a density of approximately 2.0×10⁶ cells/well. Culture dishes are pre-coated with poly-D-lysine.

After 1 hour, the medium is removed and fresh medium added (1.5 ml/well). One group of cultures is treated chronically with the compound at a concentration of 1 μM. Untreated cultures served as controls. The medium is changed every other day and antimitotics and antibiotics are not used at any stage.

After 7 days in culture, cultures are then immunostained for tyrosine hydroxylase (TH). Briefly, the cells are washed in 0.05M tris-buffered saline (TBS, pH 7.4) containing 1% Triton X-100 for 3×15 minutes and incubated with 10% foetal bovine serum (FBS, Life Technologies) in TBS for 30 minutes. The cells are then incubated for 24 hours at 4° C. with monoclonal mouse anti-TH antibody (Boehringer Mannheim) diluted 1:600 in TBS with 10% FBS. After rinsing in TBS with 1% Triton X-100 for 3×15 minutes, cells are incubated for 60 minutes with biotinylated anti-mouse IgG antibody (Amersham) diluted 1:200 in TBS with 10% FBS. The cells are then washed in TBS with 1% Triton X-100 (3×15 minutes) and incubated for 60 minutes with streptavidine-peroxidase (Dako) diluted 1:200 in TBS with 10% FBS. After washing in TBS (3×15 minutes), bound antibody is visualised by treatment with 0.05% 3,3-diaminobenzidine (Sigma) in TBS containing 0.01% H₂O₂. TH-immunoreactive (ir) cells were counted manually.

Method 4

Survival of Dopaminergic Neurons from E28 Pig Ventral Mesencephali

In this test, the effect of a compound with neurotrophic activity (below: the compound) pound on the survival of dopaminergic neurons in organotypic slice cultures established from pig E28 ventral mesencephali is assessed.

Method

Ventral mesencephali (VM) are isolated from porcine embryos (E28) under sterile conditions, chopped into 400 μm slices and placed in chilled Gey's balanced salt solution (GIBCO) with glucose (6.5 mg/ml). The tissue slices are cultured by the interface culture method, originally developed by Stoppini et al. [L. Stoppini, P.A. Buchs, D. Muller A simple method for organotypic cultures of nervous tissue; J. Neurosci. Methods 1991 37 173-182].

In brief, slices are placed on semiporous membranes (Millipore, 0.3 μm; 4 slices/membrane) placed as inserts in 6-well plates (Costar) with serum containing medium (Gibco BRL). Each well contained 1 ml medium (50% Optimem, 25% horse serum, 25% Hank's balanced salt solution (all GIBCO)) supplemented with D-glucose to a final concentration of 25 mM.

At day 3, the medium was replaced by defined serum-free medium (Neurobasal medium with B27 supplement, Life Technologies). The cultures are grown in an incubator with 5% CO₂ at 36° C. for 21 days after which the sections are immunostained for TH as described in Test 2. One group of slice cultures are treated chronically with the compound at a concentration of 1 μM. Untreated cultures serves as controls. The medium is changed twice a week and antimitotics and antibiotics are not used at any stage.

Quantification of TH-ir neurons is performed on coded slides (to allow analysis by experiments “blinded” to sample identity) using an Olympus C.A.S.T. Grid system (version 1.10; Olympus, Albertslund, Denmark) composed of an Olympus BX50 microscope and a computer controlled x-y-z step motor stage. The area of the culture slice is delineated and a counting frame is randomly placed to mark the first area to be sampled. The frame is then systematically moved through the sections and the TH-ir cells counted.

Method 5

Potentiation of NGF Signal Transduction in PC12 Cells

In this test the effect of a compound with neurotrophic activity (below: the compound) on NGF-induced phosphorylation of the ERKs and the Akt kinase is assessed.

Method

Approximately 200,000 PC12 cells are plated in a 24 well plate in DMEM with 7.5% FCS and 7.5% DHS and incubated ON. The next day NGF and the compound are added to the cells and they are incubated for 24 hours after which the cells are harvested in 2×Laemmli sample buffer.

Total cell lysate is electrophoresed on 8-18% gradient SDS gels which are electroblotted to PVDF membranes. Phosphorylated ERK1 and ERK2 are immunodetected by using mouse anti-Phospho-p44/p42 MAP kinase E10 mAb (New England Biolabs #9106) and HRP-linked anti-mouse antibody. Phosphorylated Akt kinase is immunodetected by using rabbit phospho-specific Akt (Ser473) antibody (New England Biolabs # 9271) and HRP-linked anti-rabbit antibody. Bands are detected by chemilumininescence using the ECL system (Amersham).

Method 6

Stimulation of CREB Phosphorylation in Undifferentiated PC12 Cells

In this method the effect of a compound with neurotrophic activity (below: the compound) on CREB (cyclic AMP-responsive element binding protein) phosphorylation is assessed.

Method

Approximately 7.5×10⁵ PC12 cells per well are plated in collagen coated 6-well plates in DMEM with 0.75% FCS and 0.75% DHS and incubated for 48 hours. Cells are then further starved for 2 hours in serum free DMEM before stimulation with the indicated compounds for 5, 10 or 20 minutes. Cells are harvested in 1× heated sample buffer (2% SDS, 400 mM Tris, pH 8.0, 10 mM DTT and 0.25 mM Na₃VO₄) and the cell lysates are electrophoresed on 8-18% gradient SDS gels, which are electroblotted to PVDF membranes.

Phosphorylated CREB are immunodetected by using rabbit anti-Phospho-CREB (UpState Biotechnology #06-519) followed by HRP-linked anti-rabbit antibody (Amersham Life Science #NA 934). Bands are detected by chemilumininescence using the ECL system (Amersham).

Method 7

Transient Global Ischaemia in Gerbils

In this experiment, the neuroprotective effect of a compound with neurotrophic activity (below: the compound) is assessed in an animal model of transient global ischaemia.

Method

In halothane anaesthetised gerbils, right and left carotid arteries are located and occluded for 4 minutes. Animals are kept at normal body temperature before and after the operation using heating lamps. During the operation, the gerbils are placed on heating pads, the body temperature is controlled and maintained at 37±0.5° C. The test compound is dosed at specified time points after the ischaemic insult, either i.v., i.p., s.c. or p.o.

Four days later, the animals are sacrificed, brains removed and cooled to −70° C. Thereafter, the brains are sectioned in 20 μm thick sections of which 5-7 with hippocampal tissue are selected and stained with hematoxylin-eosin.

The degree of hippocampal damage is categorised into one of four groups:

Group 1: no damage in the CA₁-layer;

Group 2: the CA₁-layer partly damaged;

Group 3: the CA₁-layer completely damaged; and

Group 4: damage in more than just the CA₁-layer.

The total ischaemia score is obtained as the sum of scores in the right and left hemisphere. Kendall's tau test was used for statistic evaluation.

Method 8

In vitro Inhibiti n of ³H-dopamin (3H-DA) Uptake in Striatal Synaptosom s

In this test, the ability of a compound enhancing the dopamine activity (below: the test compound) to inhibit the uptake of ³H-dopamine in striatal synaptosomes is assessed.

Tissue preparations: Preparations are performed at 0-4° C. unless otherwise indicated. Corpi striati from male Wistar rats (150-200 g) are homogenised for 5-10 sec in 100 volumes of ice-cold 0.32M sucrose containing 1 mM pargyline using an Ultra-Turrax homogenizer. Monoamine oxidase activity will be inhibited in the presence of pargyline. The homogenate is centrifuged at 1000×g for 10 min. The resulting supernatant is then centrifuged at 27,000×g for 50 min and the supernatant is discarded. The pellet (P₂) is resuspended in oxygenated (equilibrated with an atmosphere of 96% O₂: 4% CO₂ for at least 30 min) Krebs-Ringer incubation buffer (8000 ml per g of original tissue) at pH 7.2 containing 122 mM NaCl, 0.16 mM EDTA, 4.8 mM KCl, 12.7 mM Na₂HPO₄, 3.0 mM NaH₂PO₄, 1.2 mM MgSO₄, 1 mM CaCl₂, 10 mM glucose and 1 mM ascorbic acid.

Assay: Aliquots of 4.0 ml tissue suspension are added to 100 μl of test solution and 100 μl of ³H-DA (1 nM, final concentration), mixed and incubated for 25 min at 37° C. Non-specific uptake is determined using benztropine (10 μM, final concentration). After incubation the samples are poured directly onto Whatman GF/C glass fibre filters under suction. The filters are then washed three times with 5 ml of ice-cold 0.9% (w/v) NaCl solution. The amount of radioactivity on the filters is determined by conventional liquid scintillation counting. Specific uptake is calculated as the difference between total uptake and non-specific uptake.

25-75% inhibition of specific binding must be obtained, before calculation of an IC₅₀. The test value is given as IC₅₀ (the concentration (μM) of the test compound which inhibits the specific binding of ³H-DA by 50%).

Method 9

Effect of a Compound on Striatal Dopamine in Mice Treated With MPTP

In this test, the ability of a compound enhancing the dopamine activity to increase striatal dopamine in mice treated with MPTP is assessed.

Female C57BL/ 6J mice weighing 20-25 grams (Møllegaard Breeding and Research Centre) are adapted to the laboratory for 5-7 days before the experiments with food and water freely available, room temperature 22-24° C. Light is on/off at 7 am and 6 pm, respectively. At least 5-8 mice are used per group. MPTP, HCL (RBI) is dissolved in saline just before the experiments and is tested in various doses 12.5, 25, 3×12.5 and 3×25 mg/kg sc. The test compound is tested following a pretreatment time of 30 min and 3 hrs before the subcutaneous sc injection of MPTP 25 mg/kg. The mice are sacrificed 48 hrs after the last dose of MPTP for the biochemical analysis of dopamine and its metabolites HVA and DOPAC. For biochemical analysis, the striatum of the mice is rapidly dissected out, frozen and stored at −80° C. On the day of analysis, one striatum per mouse (weighing 5-7 mg) is homogenised in 1 ml of 0.1 N Perchloric acid containing 5% EDTA. After centrifugation 14,0000×G for 30 min. 200 μl of the supernatant is filtered through a glass 0.22 μm filter. 20 μl is then injected into our ESA Coulochem II HPLC equipment with a the following column (Caracholamine HR-80 4.6 mm×80 mm 3 um Nucleosil C 18). The eluent is 10.25 g NaH₂ PO₄, 185 mg EDTA, 100 mg Octansulphonic acid. 9% methanol, pH 3.7, add 500 ml MiliQ water, filtered through 0.22 um. The Colochem ESA analytical cell is 5014A and the ESA detector has the following settings: E₂-175 mV, run time 16 min. for the elution of dopamine, DOPAC and HVA (DOPAC=4.3 min; dopamine=6.4 min and HVA=12.7 min.). The autoinjector SHIMADZY sil-10A has the following settings; injection vol. 20 μl, 16 min analysis, temp 4° C. Flow rate from the pump is 0.80 ml/min. The analyses are calibrated with standards of 3 pM of dopamine, HVA and DOPAC for each 12 analysis run and are compared with our standard curves.

Method 10

Effect of a Compound on Extracellular Dopamine Measured by Microdialysis

In this test, the ability of a compound enhancing the dopamine activity to increase dopamine in various brain regions is assessed.

Male SPF Mol Wistar rats weighing 300-350 g are obtained from Møllegaard Breeding and Research Centre and housed in standard Macrolon cages sized 24×36×18 cm for at least 5 days under standard conditions at a temperature of 23±2° C. and a humidity of 60%±10%, and a 12 hr light and dark cycle. The rats are housed in groups of two with food and water freely available ad libitum. For microdialysis, the rat is placed in a stereotaxic instrument under halothan anesthesia using 1½% halothan, 20% oxygen and 80% nitrous oxide. The rectal temperature is monitored and maintained at 37.0±1° C. during the experimental period using a heating pad (CMA 150 Carnegie Medicin). A small hole is drilled to allow a vertical probe (CMA/123), to be stereotaxically implanted into the right striatum, using the following coordinates relative to bregma: AP +1 mm; L 3 mm; DV −6 mm. The probes for the nucleus accumbens (CMA 122) is implanted vertical at the following coordinates: AP +2.4, L1.4 and DV −8 mm. Similar experiments are performed with probes implanted into the nucleus accumbens in non anesthetised freely moving animals. These experiments are performed 48 hr after surgery during the daylight period in animals housed individually in plastic cages with food and water availabl ad libitum. In all cases, the injection sites are confirmed histologically according to the atlas of Paxinos and Watson.

After an initial 2 hr period, samples of dialysate are collected from halothane anaesthetised rats. The dosing of a test compound to these rats are usually initiated after the collection of 3 base line analyses. Dopamine and its metabolites are rapidly frozen to −18° C. and then analyzed as soon as possible thereafter. The dialysis probe is perfused at a rate of 2 μl/min (by a CMA/100 microperfusion pump) with Ringer's solution (147 mM NaCl, 4 mM KCl, 2.3 mM CaCl) i.e. Ringer's solution (NaCl 4.3 g, KCl 150 mg, CaCl₂ 110.3 mg ad 500 ml) adjusted to pH 6.5 with 2 mM sodium phosphate buffer. The Ringer solution is filtered before use through Millipore glass filters (0.22 μm). The dialysate fractions (40 μl) are collected at 20 min intervals and then injected into the HPLC system. The concentration of dopamine (DA), dihydroxy phenyl acetic acid (DOPAC), homovanillic acid (HVA) and 5-hydroxy indolacetic acid (5-HIAA) are determined by high-performance liquid chromatography with electrochemical detection (HPLC-ED). The column is a reverse-phase liquid chromatography Catecholamine 3 μm ESA column at 23° C., the mobile phase consisting of 0.055 M sodium acetate with 0.1 nM octanesulfonic acid, 0.01 mM Na EDTA, and 10% methanol pH 3.7 adjusted with glacial acetic acid). The mobil phase is delivered by a HPLC pump (ESA) at 0.55 ml/min. Electrochemical detection is accomplished using an amperometric detector (Antec) with a glassy carbon electrode (0.8 V an Ag/AgCl reference) or a coloumetric detector (Choulochem II model ESA; with a high sensitivity analytical cell (5011). (0,4V an Ag/AgCl reference). Chromatograms are recorded by an integrator. The data are calculated as percent change of the basal concentration, the 100% value being defined as the average of the last 3 pretreatment values for each rat. The mean percentage values are then calculated for each 20 min sample for the rats in each group of treatment.

Method 11

Effect of a Compound on Degeneration of Nigral Dopaminergic Neurons After Striatal 6-OHDA Lesion

In this test, the ability of a compound enhancing the dopamine activity to increase the number of surviving dopamine neurons in the substantia nigra after a striatal 6-OHDA lesion is assessed.

FluoroGold (0.2% solution in 0.9% NaCl, 0.2 μl/side) is injected bilaterally in the striatum of halothane anaesthetised female Sprague Dawley rats weighing approximately 200-250 g with a 10 μl Hamilton syringe. The following coordinates are used: AP=+1.0 mm, ML=+/−3.0 mm, DV=−5.0 mm, tooth bar=0.0. After 1 week, 6-OHDA (20 μg free base dissolved in 0.9% NaCl supplemented with 0.02% ascorbic acid) is injected unilaterally in the striatum with a glass capillary using the following coordinates: AP=+1.0 mm, ML=−3.0 mm, DV=−5.0 mm, tooth bar=0.0. Test compound or vehicle is administered i.p., p.o., s.c. or i.v. either daily or at specified time points starting after the 6-OHDA injection. Three weeks after the 6-OHDA injection, the rotational behaviour of the 6-OHDA lesioned animals after administration of amphetamine (2.5 mg/kg i.p.) is monitored in automated rotometer bowls. Three to four weeks after the 6-OHDA injection, the rats are deeply anaesthetised and transcardially perfused with 0.9% NaCl for 1 min followed by 4% paraformaldehyde in 0.1 M phosphate buffer for 6 min. Brains are dissected out and postfixed for three to six hours in formalin and then transferred to 25% sucrose in 0.1 M phosphate buffer for 48 hours. Series of 40 μm sections are obtained by freezing microtomy through the striatum and the substantia nigra. Sections are stained for tyrosine hydroxylase (TH) immuno activity using mouse-anti-TH (Chemicon, #MAB 318). Sections are rinsed in KPBS and thereafter quenched using 10% methanol+3% hydrogenperoxide in KPBS. Preincubation for one hour in 2% normal horse serum (NHS)+0.3% Triton X-100 in KPBS. Thereafter, sections are incubated inmouse-anti-TH (Chemicon, #MAB 318) 1:2000+2% NHS+0.3% Triton X-100 in KPBS over night. After rinsing in KPBS, sections are incubated in biotinylated horse-anti-mouse (Vector) 1:200 in 0.3% Triton in KPBS for 2 hours. After rinsing in KPBS, immunoreativity is visualised by the ABC reaction (Vector Kit) followed by DAB staining. Surviving dopaminergic neurons in the 6-OHDA lesioned and intact sides are quantified blindly by stereologically counting the number of retrogradely labelled neurons in the substantia nigra displaying fluorogold fluorescence and by counting the number of neurons displaying TH immunoreactivity. In some cases, the degree of neuronal survival is estimated by assigning a score from one to five to each section depending on the fraction of surviving dopaminergic cells as estimated blindly by observing sections processed for fluorogold flourescence and/or TH immunohistochemistry. The score “1” is assigned to sections in which all neurons survive and are morphologically indistinguishable from non-lesioned neurons whereas the score “5” is assigned to sections in which no neurons survive in the 6-OHDA lesioned side.

Method 12

Inhibition of In Vitro [³H]WIN 35428 Binding to the Dopamine Transporter in Rat Striatal Synaptosomes

In this method the ability of test compounds to inhibit the binding of WIN 35428 to the dopamine transporter in vitro is assessed.

Tissue preparation: Striata from male Wistar rats (150-200 g) are homogenized for 5-10 sec in 10 ml NaH₂PO₄ (50 mM, pH 7.4) using an Ultra-Turrax homogenizer. The suspension is centrifuged at 27,000×g for 15 min. The supernatant is discarded and the pellet is resuspended in phosphate buffer (1000 ml per g of original tissue) and used for binding assays.

Binding assays: Aliquots of 0.5 ml tissue suspension are added to 0.025 ml of test solution and 0.025 ml of [³H]WIN 35428 (1 nM, final concentration), mixed and incubated for 60 min at 2° C. Non-specific binding is determined using cocaine (30 μM, final concentration).

After incubation 5 ml of ice-cold buffer is added to the samples and poured directly onto Whatman GF/C glass fiber filters (for the [³H]GBR 12935 assay the filters are presoaked in 0.1% PEI for at least 20 min) under suction and immediately washed with 5 ml ice-cold buffer. The amount of radioactivity on the filters is determined by conventional liquid scintillation counting using a Tri-carb liquid scintillation analyzer (model 1600CA; Packard, USA). Specific binding is calculated as the difference between total binding and non-specific binding.

Data analysis: The test value is given as an IC₅₀ (the concentration (PM) of the test substance which inhibits the specific binding of [³H]ligand by 50%). Five to nine concentrations are been used to determine the inhibition curves from which the IC₅₀ values are determined. If a full curve is not available a 25-75% inhibition of specific binding must be obtained, before calculation of an IC₅₀.

$${IC}_{50} = ( applied test substance concentration , µM ) \times \frac{1}{( \frac{{C}_{o}}{{C}_{x}} - 1 )}$$

where Cₒ is specific binding in control assays and Cₓ is the specific binding in the test assay. (The calculations assume normal mass-action kinetics).

Method 13

Survival of Embryonic Rat Dopaminergic Neurons

In this experiment the effect of a test compound on the survival of dopaminergic neurons in dissociated cultures established from rat E14 ventral mesencephali (VM) is assessed.

Method

Embryonic rat brains (Wistar; E14) were isolated under sterile conditions placed in chilled Gey's balanced salt solution (GIBCO) with glucose (6.5 mg/ml).

The ventral mesencephali were dissected out, cut into small tissue pieces, placed in Neurobasal medium with B27 supplement and gently pressed through a 80 μm Nitex filter. The cells were counted using a hemocytometer and plated in a 6 well multi-dish at a density of approximately 2.0×10⁶ cells/well. Culture dishes were pre-coated with poly-D-lysine.

After 1 hour, the medium was removed and fresh medium added (1.5 ml/well). One group of cultures was treated chronically with test compound at a concentration of 1 μM. Untreated cultures served as controls. The medium was changed every other day and antimitotics and antibiotics were not used at any stage.

After 7 days in culture, cultures were then immunostained for tyrosine hydroxylase (TH). Briefly, the cells were washed in 0.05M tris-buffered saline (TBS, pH 7.4) containing 1% Triton X-100 for 3×15 minutes and incubated with 10% foetal bovine serum (FBS, Life Technologies) in TBS for 30 minutes. The cells were then incubated for 24 hours at 4° C. with monoclonal mouse anti-TH antibody (Boehringer Mannheim) diluted 1:600 in TBS with 10% FBS. After rinsing in TBS with 1% Triton X-100 for 3×15 minutes, cells were incubated for 60 minutes with biotinylated anti-mouse IgG antibody (Amersham) diluted 1:200 in TBS with 10% FBS. The cells were then washed in TBS with 1% Triton X-100 (3×15 minutes) and incubated for 60 minutes with streptavidine-peroxidase (Dako) diluted 1:200 in TBS with 10% FBS. After washing in TBS (3×15 minutes), bound antibody was visualised by treatment with 0.05% 3,3-diaminobenzidine (Sigma) in TBS containing 0.01% H₂O₂. TH-immunoreactive (ir) cells were counted manually.

Method 14

Effect of a Compound on Catalepsy

In this test, the ability of a test compound to influence catalepsy induced by haloperidol is assessed.

Male wistar rats weighing 200-250 g are housed in cages of four rats with food and water ad lib and with a 12 hour light cucle. Test compound or vehicle is administered i.p., p.o., s.c. or i.v. at specified time points before haloperidole administration (0.1 mg/kg s.c.). For each dose levels 6 rats are tested. Testing for catalepsy is performed at 15 min intervals including 4 tests performed consecutively, in each test evaluating the intensity of catalepsy for 10 sec.

1) A vertical wire netting (40×40 cm high). The meshes (openings) of the netting are approximately 1×2 cm.

2) A horizontal bar 9 cm above the floor

3) A 9 cm high block (bar)

4) A 3 cm high block (cork)

The rat is placed in the middle of the vertical wire netting, then on the horizontal bar in an extended position supporting the forelegs on the bar. The intensity of catalepsy is evaluated according to a criterion of 10 sec of total immobility for a score of 2. Minor movements of the head or the body give the score of 1 and a score of 0 is given, if the rat shows no syndrome. The rats are then tested after the bar test, whether or not they were willing to sit with the left or right foreleg placed first on the 9 and then on the 3 cm block for a duration of 10 sec. The maximum score for all 4 tests is thus a total of 8.

### BRIEF DESCRIPTION OF THE DRAWINGS

The present invention is further illustrated with reference to the accompanying drawings, in which:

FIG. 1 illustrates the effect of compound a on the survival of PC12 cells deprived of serum and NGF;

FIG. 2 illustrates the effect of compound a on the stimulation of neutite outgrowth in PC12 cells;

FIG. 3 illustrates the effect of compound b on catalepsy induced by haloperidol; and

FIG. 4 illustrates the effect of compound a on the hippocampal damage in a gerbil model of ischemia.

### EXAMPLES

The invention is further illustrated with reference to the following examples, which are not intended to be in any way limiting to the scope of the invention as claimed.

### Example 1

The compound 5-(4-Chlorophenyl)-6,7,8,9-tetrahydro-1-H-pyrrolo[3.2-h]naphthalene-2,3-dione-3-oxime (compound a) was tested for in vitro inhibition of [³H]WIN 35428 binding according to test method 12.

The result of the test was an IC₅₀ value for compound a of 0.14 μM.

### Example 2

Compound a was tested for in vitro inhibition of [³H]-DA according to test method 8.

The result of the test was an IC₅₀ value for compound a of 34 nM.

### Example 3

Compound a was tested for survival of embryonic rat dopaminergic neurons according to test method 13.

The results of this experiment are presented in the table below:

| Concentration of | Inhibition of |
|---|---|
| compound a | cell loss |
| 0.3 μM | 74% |
| 1.0 μM | 62% |
| 3.0 μM | 17% |

### Example 4

Compound a was tested in a PC12 cells survival assay according to test method 2.

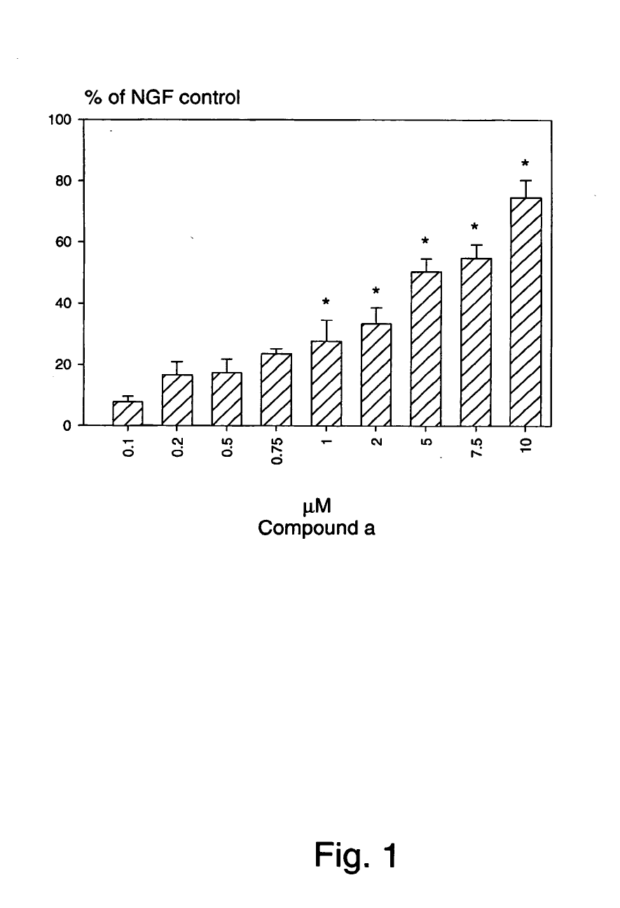

The results of the experiment are presented in FIG. 1.

Cell viability was estimated by MTS reduction, and data are expressed as % of the NGF response, meaning the value determined with 3 nM NGF corrected for the values in parallel serum-free cultures. Data shown are the means±S.E.M. (n=6) from a representative experiment, and * indicates a significant difference from untreated cultures (P<0.05, one way ANOVA, Dunnett's Method).

### Example 5

Compound a was tested for stimilation of neurite outgrowth in PC12 cells according to test method 1.

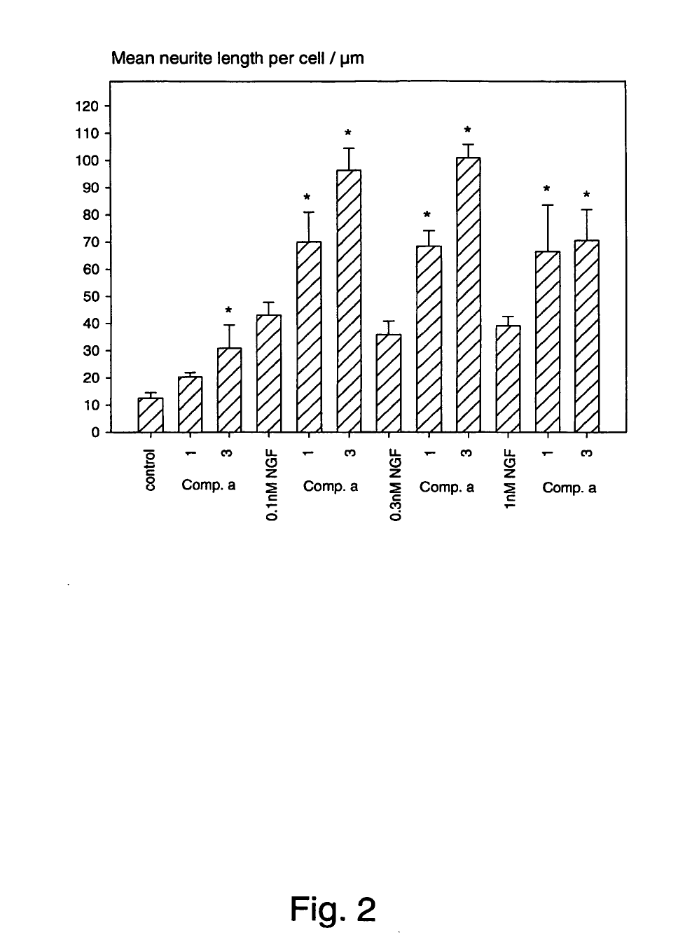

The results of the experiment are presented in FIG. 2.

Cells were incubated with 0, 1 or 3 μM of compound a and the indicated concentrations of NGF for 48 hours. Neurite outgrowth was quantified using unbiased 2D stereology using the CAST-grid system connected to an Olympus BH-2 microscope. Data are expressed as the average neurite length±S.E.M, and * indicates a significant difference from the respective control without compound a (P<0.05, one way ANOVA, all pair wise multiple comparison procedures, Student-Newman-Keuls Method).

### Example 6

The compound 5-(4-Chlorophenyl)-6,7,8,9-tetrahydro-1-H-pyrrolo[3.2-h]naphthalene-2,3-dione-3-O-(4-hydroxybutyric acid-2-yl)oxime (compound b) was tested for effect on catalepsy by haloperidol (0.1 mg/kg s.c.) according to test method 14. Compound b was dosed perorally 2 h before haloperidol treatment.

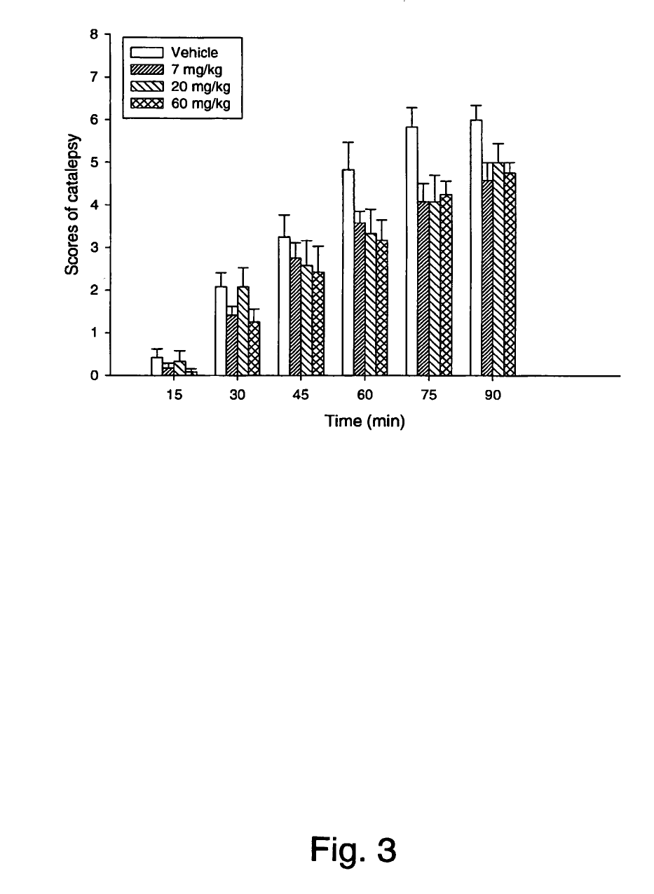

The results of the experiment are presented in FIG. 3.

### Example 7

Compound a was tested in the gerbil model of global ischemia according to test method 7.

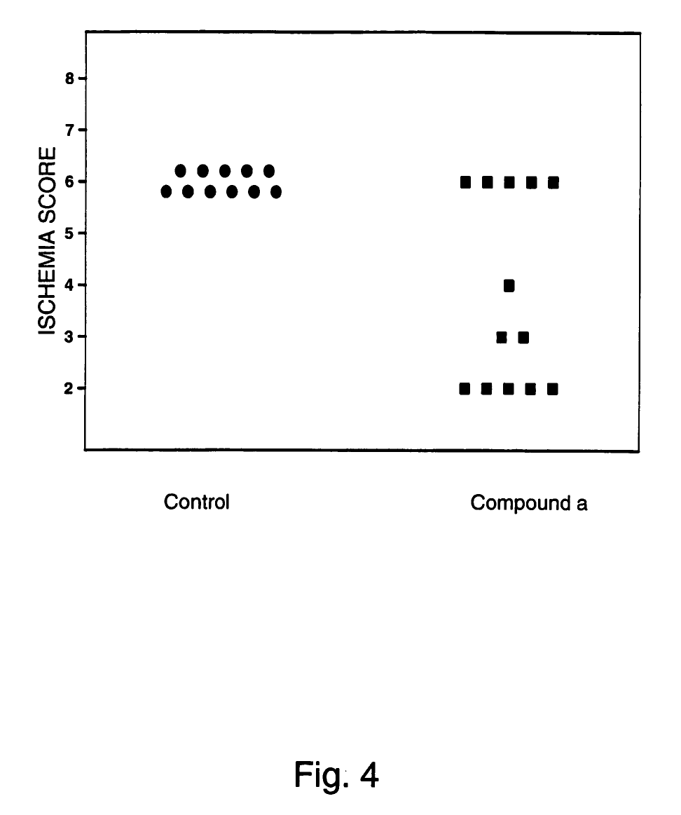

The results of the experiment are presented in FIG. 4.

Animals received compound a (10 mg/kg ip.) 2 minutes after the ischemic insult and the following day. Four days later, the animals were sacrificed, and 20 mm thick brains slices were stained with hematoxylin-eosin. The degree of hippocampal damage was categorized into one of four groups as described in the test method. The total ischemia score was obtained as the sum of scores in the right- and left hemisphere, and Kendall's tau test was used for statistic evaluation (p=0.01).

1. A pharmaceutical composition comprising a therapeutically effective amount of at least one compound with neurotrophic activity and at least one compound enhancing the dopamine activity, together with at least one pharmaceutically-acceptable carrier or diluent.

2. The pharmaceutical composition of claim 1, wherein the compound enhancing the dopamine activity is a monoamine reuptake inhibitor.

3. The pharmaceutical composition of claims 1 or 2, wherein the compound with neurotrophic activity and the compound enhancing the dopamine activity are not the same compound.

4. The pharmaceutical composition of claim 3, wherein the compound with neurotrophic activity is GDNF and the compound enhancing the dopamine activity is Bupropion.

5. The pharmaceutical composition of claim 3, wherein the compound with neurotrophic activity is GDNF and the compound enhancing the dopamine activity is Nomifensine.

6. The pharmaceutical composition of claims 1 or 2, wherein the compound with neurotrophic activity and the compound enhancing the dopamine activity are the same compound.

7. The pharmaceutical composition of claim 6, wherein the compound is
5-(4-Chlorophenyl)-8-methyl-6,7,8,9-tetrahydro-1-H-pyrrolo[3.2-h]isoquinoline-2,3-dione-3-oxime;
5-(4-Chlorophenyl)-6,7,8,9-tetrahydro-1-H-pyrrolo[3.2-h]naphthalene-2,3-dione-3-oxime;
5-(4-Chlorophenyl)-6,7,8,9-tetrahydro-1-H-pyrrolo[3.2-h]naphthalene-2,3-dione-3-O-(4-hydroxybutyric acid-2-yl)oxime; or
or pharmaceutically acceptable salts thereof.

8. The pharmaceutical composition of any one of the claims 1-3, wher in the compound with neurotrophic activity is a compound of claim 14 or a compound selected from
[N-(2-Hydroxy-4-methoxyphenyl)-N′-(3-trifluoromethylphenyl)]urea;
5-(4-Chlorophenyl)-8-methyl-6,7,8,9-tetrahydro-1-H-pyrrolo[3.2-h]isoquinoline-2,3-dione-3-oxime;
5-(4-Chlorophenyl)-6,7,8,9-tetrahydro-1-H-pyrrolo[3.2-h]naphthalene-2,3-dione-3-oxime; or
5-(4-Chlorophenyl)-6,7,8,9-tetrahydro-1-H-pyrrolo[3.2-h]naphthalene-2,3-dione-3-O-(4-hydroxybutyric acid-2-yl)oxime;
or pharmaceutically acceptable salts thereof.

9. The pharmaceutical composition of any one of the claims 1-8, for use in the treatment, prevention or alleviation of Parkinson's disease in a subject.

10. A combination of at least one compound with neurotrophic activity and at least one compound enhancing the dopamine activity for use as a therapeutic agent.

11. Use of at least one compound with neurotrophic activity and at least one compound enhancing the dopamine activity for the manufacture of a medicament for the treatment, prevention or alleviation of Parkinson's disease in a subject.

12. A method of treatment, prevention or alleviation of Parkinson's disease in a subject, which method comprises administering to said subject a therapeutically effective combination of at least one compound with neurotrophic activity and at least one monoamine reuptake inhibitor.

13. A kit of parts comprising at least one compound with neurotrophic activity and at least one compound enhancing the dopamine activity.

14. A novel compound being
[N-(5-Chloro-2-hydroxyphenyl)-N′-(3-nitrophenyl)]urea;
[N-(2-Hydroxy-4-methoxyphenyl)-N′-phenyl]urea;
[N-(3-Aminophenyl)-N′-(2-hydroxy-4-methoxyphenyl)]urea;
[N-(3-Chlorophenyl)-N′-(2-hydroxy-4-methoxyphenyl)]urea;
[N-(2-Hydroxy-4-methoxyphenyl)-N′-(4-trifluoromethylphenyl)]urea;
[N-(2-Hydroxy-4-methoxyphenyl)-N′-(3-nitrophenyl)]urea;
[N-(4-Chlorophenyl)-N′-(2-hydroxy-4-methoxyphenyl)]urea;
[N-(2-Hydroxy-4-methoxyphenyl)-N′-(2-trifluoromethylphenyl)]urea;
[N-(4-Chloro-2-hydroxy-5-methylphenyl)-N′-(phenyl)]urea;
[N-(3-Bromophenyl)-N′-(2-hydroxy-4-methoxyphenyl)]urea;
[N-(3-Fluorophenyl)-N′-(2-hydroxy-4-methoxyphenyl)]urea;
[N-(2-Chloro-5-trifluoromethylphenyl)-N′-(2-hydroxy-4-methoxyphenyl)]urea;
[N-(3,5-Di-(trifluoromethyl)-phenyl)-N′-(2-hydroxy-4-methoxyphenyl)]urea;
[N-(5-Bromo-2-hydroxyphenyl)-N′-(3-trifluoromethylphenyl)]urea;
[N-(2-Hydroxy-4-methoxyphenyl)-N′-(3-methoxycarbonylphenyl)]urea;
[N-(2-Hydroxy-4-methoxyphenyl)-N′-(3-methylcarbonylphenyl)]urea;
[N-(3-Cyanophenyl)-N′-(2-hydroxy-4-methoxyphenyl)]urea;
[N-(5-Ethoxy-2-hydroxy-4-(N-morpholinyl)phenyl)-N′-(3-trifluoromethylphenyl)]urea;
or pharmaceutically acceptable salts thereof.

15. A method for screening a compound for activity in the treatment, prevention or alleviation of Parkinson's disease in a subject, which method comprises the following steps:
measuring the ability of the compound to enhance the dopamine activity;
measuring the neurotrophic activity of the compound.

16. A method for identification of a compound active in the treatment, prevention or alleviation of Parkinson's disease in a subject, which method comprises the following steps:
screening compounds for the ability to enhance the dopamine activity and for the neurotrophic activity:
selecting a compound with ability to enhance the dopamine activity and with neurotrophic activity.
